(19) United States
(12) Patent Application Publication
(10) Pub. No.: US 2005/0175551 A1
(43) Pub. Date: Aug. 11, 2005

(54) Silicon modified polyamide material useful for oral care

(76) Inventors: Heng Cai, Yardley, PA (US); Marian N. Holerca, Highland Park, NJ (US); Arthur Chen, East Brunswick, NJ (US); Van Canady, Princeton, NJ (US); John Afflitto, Brookside, NJ (US)

Correspondence Address: COLGATE-PALMOLIVE COMPANY, 909 RIVER ROAD, PISCATAWAY, NJ, 08855, US

(21) Appl. No.: 10/516,874

(22) PCT Filed: Oct. 24, 2003
(86) PCT No.: PCT/US03/33914

Related U.S. Application Data
(60) Provisional application No. 60/421,037, filed on Oct. 24, 2002.

(51) Int. Cl. A61K 7/16

(52) U.S. Cl. 424/49

(57) ABSTRACT

The invention is directed to a new material useful in oral care products such as dental floss or as a toothbrush bristle. The material is a silicon-modified polyamide blended with a different polyamide material. The preferred different polyamide material is the conventional material used to make bristles of toothbrushes. The different polyamide material is preferably a nylon material.

### BACKGROUND OF THE INVENTION

The invention is drawn to a novel silicon modified polyamide material that is useful in the oral care field. For example, the material can be used as filaments that can be used as toothbrush bristles, interdental brushes, paint brush bristles such as for a cleaning or whitening system, dental floss tape (string), dental strips or tube applicators etc.

Several patents describe bristles of tooth brushes. For example, U.S. Pat. No.6,327,736 discloses making bristles for a toothbrush from a plastic material chosen from a group of plastics consisting of polyamides and polyesters. Polyamides include nylon. Other patents that discuss making bristles and/or dental floss are U.S. Pat. No. 6,475,553

Manufacturers of nylon bristle toothbrushes have provided in the past, a variety of toothbrushes designated as “soft,” “medium,” and/or “hard” to indicate the stiffness of the bristles. For a given thermoplastic polymeric composition, one factor, which predominantly determines bristle stiffness, is the diameter of the individual bristles. For example, with nylon 6,12 the “soft” bristles typically have a diameter between 0.007 and 0.008 inches; “medium” bristles have a diameter between 0.008 and 0.009 inches and “hard” bristles have a diameter greater than about 0.010 inches. Polybutylene terephtalate bristles are typically about 0.001 to 0.002 inches smaller in diameter due to the greater wet stiffness of this material over that of nylon 6,12. For all bristles used in toothbrushes, there is generally a manufacturing or grading tolerance of about +/−0.0005 inches.

Soft, medium, and hard bristles are all effective in cleaning teeth while soft bristles are more gentle on the gum and soft tissues of the oral cavity, medium and hard bristles show stronger brushing actions on the tooth surfaces.

U.S. Pat. No. 6,138,314 discloses making bristles for a toothbrush from polyamide materials such as nylon materials. The synthetic polyamides useful in the bristles of toothbrushes includes those which are of sufficient molecular weight to be fiber-forming such as: polycaprolactam, polyhexamethylene adipamide, polyhexamethylene sebacamide, the polyamide formed from 1,4,(cis)cyclohexane-bis(methylamine) and adipic acid (see U.S. Pat. No. 3,012,994); the polyamide from m-xylene diamine and adipic acid (see U.S. Pat. No. 2,916,475); the polyamide from 3,5 dimethyl hexamethylene diamine and terephthalic acid (see U.S. Pat. No. 2,752,358); the polyamide from 2,5 dimethyl piperazine and adipyl chloride (see U.S. Pat. No. 3,143,527). See also U.S. Pat. No. 2,152,606. The preferred polyamides are polyhexamethylene adipamide; and polyhexamethylene sebacamide. In general, the number average molecular weight of the polymer used for these bristles should be in excess of 10,000 and preferably greater than 30,000 to provide the strength and stiffness needed in a toothbrush bristle. The commercial polyamides preferred include nylon 6, nylon 6,6; nylon 6,10 and nylon 6,12.

U.S. Pat. No. 5,560,377 discloses making dental floss from a composite of a multifilament yarn bonded to an extruded monofilament. Both elements are made of polymer compounds, preferably nylon, to provide desired ease of use of the monofilament as a leader to pass the implement easily between the teeth or under bridges while the multifilament yarn can be provided in looped embodiments, as a bush element or in the form of one or more tails thus providing superior flossing action when passed between the teeth or under bridges.

U.S. Pat. No. 6,604,534 (“'534 patent”). discusses monofilament tapes and monofilament interproximal devices. The '534 patent discusses the following patents as being relevant to monofilament interproximal devices: U.S. Pat. Nos. Re 35,439; 3,800,812; 4,974,615; 5,760,117; 5,433,226; 5,479,952; 5,503,842; 5,755,243; 5,845,652; 5,884,639; 5,918,609; 5,962,572; 5,998,431; 6,003,525; 6,083,208; 6,148,830; 6,161,555; and 6,027,592, the disclosures of which are hereby incorporated herein by reference. These dental tapes generally have serious shortcomings in gentleness, in delivering coatings during flossing and in being handled easily and conveniently during flossing.

U.S. Pat. No. 6,051,216 discloses the use of cosmetic compositions containing siloxane based polyamides as thickening agents.

### SUMMARY OF THE INVENTION

It is an object of the present invention to develop a new material useful in the oral care industry.

It is another object to develop a new material useful as a filament, preferably a monofilament or bristle used in the oral care industry, in particular in an oral care product. An oral care product would be any known product that is used in the mouth or for the mouth. For example, an oral care product can be but is not limited to a toothbrush, paintbrush, dental floss tape (string), dental strips or tube applicators dental tape etc. The bristle can be used for a toothbrush that can be used on a manual non-electric or electric toothbrush that has superior properties with respect to sensory and antibacterial.

Another object of the present invention to develop a new material that can be used for dental floss that has superior properties with respect to sensory and antibacterial.

Another object of the present invention is to use the bristle on a paint brush that can be used to apply whitening compositions to the teeth (bleaching the teeth) or a cleaning composition to the teeth.

One aspect of the invention is a filament which comprises a silicon-modified polyamide material blended with a different polyamide material. The different polyamide material can be the commercial or prior art material that is used for dental floss or toothbrush bristles, such as the materials discussed above, which include nylon. The filament can be used in dental floss, dental tape, a toothbrush bristle or a paint brush bristle. The paint brush can be used in an application to place a whitening composition such as a bleaching composition on the teeth to make the teeth whiter.

### DETAILED DESCRIPTION OF THE INVENTION

We have found a new material used for making filaments that can be used in any oral care product. The material can be used as a filament such as but not limited to tooth brushes, dental floss, dental tape, and paint brushes. The paint brushes can be used to apply a cleaning, whitening or any other composition to the mouth and preferably the teeth. The filament can be substituted for any commercial oral care product that contains a filament. The commercial oral care product would be made in any known manner.

We have found that the incorporation of silicone into polyamides results in new physical and chemical properties that modify and improve the performance of the polyamides. The modification means changing the hydrophilicity or Hydrophobicity of the polyamides or changing the lubricity of the polyamides. Improvements can also be introduced by the functionalities attached to the silicones, which can exhibit antibacterial properties or affinity for certain metal ions, quaternary ammonium ions, fluoride ions or benefical enzymes. Changes in the physical properties of polyamides include altering the crystallinity of polyamides, for example, reducing the crystallinity of the polyamide for example, Nylon 6,6 to a level similar to that of Nylon 6,12. These properties, when present in nylon resins can provide superior benefits for making toothbrush bristles and dental floss.

Siliconized polyamides, contain both silicone moieties and amide moieties in the backbone. The silicone component comes with unique flexibility, stability and surface tension properties, while the amide moieties make the molecule compatible with other polyamides, such as, the ones described in the background of the invention which are not limited to nylon. As discussed above, polyamides, such as nylon are a key material for toothbrush and dental floss technology. It is conceived that the siliconized polyamide will modify the other polyamide structure, such as the polyamide structures discussed above in the background of the invention section which includes, nylon structure when mixed, and particularly the surface structure of the polyamide such as nylon so that the modified polyamide (i.e. nylon) for bristles and dental floss are softer, and more lubricious. This would provide a significant benefit to reduce toothbrush trauma to the gingival, which is a major cause of gingival recession. More importantly, it is also known that silicone has very low surface tension, and such a property would be ideal for reducing bacterial adhesion and accumulation on the surfaces of toothbrush bristles (anti-attachment), a pro-hygenic effect which would be viewed as desirable by the consumer. Therefore, siliconized polyamide modified polyamides such as, but not limited to nylons are superior materials for toothbrush bristle and dental floss.

In addition, the chemistry involved in developing siliconized polymides can also be used to graft other silicones such as cationic silicones or non-silicone moieties such as quaternary ammonium ions to nylon to achieve superior properties desired for toothbrush bristles and dental floss. Siliconized polyamides used according to this invention described in detail in U.S. Pat. No. 6,051,216 (“'216 patent”) which is incorporated by reference in its entirety for all useful purposes. The '216 patent claims a silicon-modified polyamide comprises
a) 0.5-80 percent by weight based on the total weight of the composition of at least one siloxane-based polyamide;
(b) 5-95 percent by weight silicone fluid; and
(c) if (a)+(b) do not equal 100 percent, then a portion of solvent sufficient to make 100 percent, wherein:

the siloxane-based polyamide is formed from units of Formula A 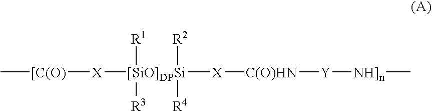
where: (1) n is a number selected from the group consisting of 1-500, where n is the number of units in the polyamide;
(2) DP is an average value for degree of polymerization of a siloxane portion of the polyamide and is selected from the group consisting of 1-700;
(3) X is a linear or branched chain alkylene having 1-30 carbons;
(4) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl; and
(5) Y is selected from the group consisting of linear or branched chain alkylenes having 1-40 carbons, wherein the alkylene group itself may optionally be substituted by at least one member selected from the group consisting of
(i) hydroxy;
(ii) C₃ -C₈ cycloallcane;
(iii) 1-3 members selected independently from the group consisting of C₁-C₃ alkyls; phenyl optionally substituted by 1-3 members independently selected from the group consisting of C₁-C₃ alkyls;
(iv) C₁-C₃ alkyl hydroxy; and
(v) C₁-C₆ alkyl amine;
or Y may be Z where Z=R²⁰ T(R²²)R²¹ where each of R²⁰, R²¹ and R²³ is independently selected from the group consisting of linear and branched C₁-C₁₀ alkyl or alkylene groups; and T is selected from the group consisting of CR, where R is selected from the group consisting of hydrogen, the group consisting of the group defined for R¹-R⁴; and a trivalent atom selected from the group consisting of N, P and Al; and wherein each value for X, Y, DP and R¹-R⁴ may be the same or different for each unit in the polyamide. In addition, the siloxane-based polyamide preferably (1) comprise both siloxane groups and amide groups to thicken compositions containing silicone fluids (volatile and/or non-volatile silicone fluids); (2) are non-flowable solids at room temperature; and (3) dissolve in a fluid which contains silicone at a temperature of 25-160 degrees C. to form a translucent or clear solution at a temperature in this range.

When making a toothbrush, the filaments lie in close proximity to each other as the result of winding or braiding the filaments, or generally as the result of stranding the filaments. The surface area of the filaments can be subjected to partial dissolving by means of chemical agents. In this manner, the filaments merge completely together and any cavities still remaining in the center of the wound or braided bristle are closed. This process can be supported by any existing or selectively variable tensile stress acting on the filaments. Altogether a cavity-free bristle is thus produced, offering bacteria or other germs no possibility of infiltration.

The following values have proven to be especially suitable in particular for an electric toothbrush: three or four filaments per bristle are used, the diameter of the individual filaments lies between approximately 0.0762 mm and 0.127 mm, approximately, and the winding or braiding of a bristle is repeated after every 1.0 mm approximately to 3.0 mm, approximately.

The bristles may be formed by melt extruding various thermoplastic polymeric materials through appropriately shaped extrusion orifices in various dies following various processes such as described in U.S. Pat. Nos. 2,226,529 and 2,418,482; 3,745,061; 3,238,553; 3,595,952; 4,279,053; and French Patent No. 2,125,920.

The tufting, cutting, stapling, etc., of the bristles is performed by processes known in the art; for instance as described in U.S. Pat. Nos. 4,441,227; 4,688,857; 979,782; 5,274,873; 5,335,389; and 5,511,275, the disclosures of which are hereby incorporated herein by reference.

Experiments with blends of siliconized polyamides and polyamides such as nylon have indicated that the two polymers are miscible above their melting temperature, and they are compatible at the macro-scale at room temperature. Optical microscopy was employed to demonstrate that at the micro-scale, the silicone component occupies a specific volume fraction within the resulting tertiary structure of filaments. This demonstrated the role of silicone as a modifier of nylon.

The following additional examples were conducted:

Masterbatches with 20% polyamide additive in Nylon 6,6 and Nylon 6,12 were produced. The masterbatches did not contain anything else. Masterbatches had to be produced carefully to ensure a good dispersion of the low-melting additive in the Nylons. Fibers were produced using Nylon 6,6 and Nylon 6,12 to identify correct processing conditions for both. In general Nylon 6,6 was processed around 285-290° C. in the fiber line and Nylon 6,12 was processed around 235-240° C. Fibers were prepared using a round die containing 41 die holes. Fibers are “flat yarn” and are 6-8 denier per filament (250-325 total denier). Fibers were then prepared using correct letdown ratios of the masterbatch in appropriate resins. The following data gives average values from tensile and DSC data.

Nylon 6,6 fibers

| Additive | | Elongation | | | Melting |
|---|---|---|---|---|---|
| level | Tenacity* | at peak | Elongation | Crystallinity | point |
| (% w/w) | grams/tex | (%) | at break (%) | (%) | deg. C. |
| 0 | 17.64 | 353 | 359 | 26.6 | 265.3 |
| 1 | 14.84 | 429.4 | 599.8 | 27 | 265.9 |
| 3 | 17.01 | 217.1 | 392.1 | 22.2 | 266.8 |

Nylon 6,12 fibers

| Additive | | Elongation | | | Melting |
|---|---|---|---|---|---|
| level | Tenacity* | at peak | Elongation | Crystallinity | point |
| (% w/w) | grams/tex | (%) | at break (%) | (%) | deg. C. |
| 0 | 22.71 | 216.4 | 292.9 | 28 | 217.9 |
| 1 | 18.04 | 240.3 | 332.1 | 26.5 | 218.5 |
| 3 | 20.19 | 306.9 | 402.3 | 28.1 | 217.2 |

Tenacity reported is breaking tenacity

A comparison was made between mixed toothbrush bristles with siliconized polyamide coated on nylon bristles and bristles with unmodified nylon. The modified nylon showed unexpectedly improved results.

In addition, a microbiological assay of bacterial attachment to toothbrushes was performed, and the table below presents examples illustrating the new and improved bacterial anti-attachment properties of the new material. To evaluate the ability of bacteria to attach to the surface of the toothbrush bristles, an experiment was designed to quantify the number of bacteria which were left after a certain amount of rinsing time. Bacteria chosen for this experiment were selected from the many orally relevant species. One such suitable species of bacteria is Actinomyces naeslundii, a well-known early colonizer of the mouth. A bacterial suspension of A. naeslundii was first prepared by inoculating trypticase soy broth (TSB). After incubation overnight at 37.5° C., the suspension was diluted with additional TSB until the optical density (O.D.) was between 0.015 and 0.2. (The optical density in this case corresponds to the overall quantity of bacteria in the suspension.) The toothbrushes were carefully cleaned with mild detergent and then disinfected for 60 seconds in 70% ethanol. After air drying, the bristles of the brushes were dipped into the bacterial suspension for 60 seconds, rinsed in phosphate buffered saline (PBS) for 0-60 seconds, then brushed over the surface of separate agar plates. These agar plates were incubated for 2 days at 37.5° C. at which time a visual inspection was made of the colony forming units (CFU's).

At higher concentrations of bacteria, >0.05 O.D., it was not possible to count the CFU's reliably. However, in a visual inspection of these experiments there was always clearly a less dense lawn of bacterial colonies on the plate brushed with the silicone containing bristles. As the bacterial load was reduced to a OD of 0.017-0.020, two out of three experiments performed showed a clear 18-25% reduction in bacteria in toothbrushes with Nylon 6,6+3% silicone-modified polyamide, relative to Nylon 6,12.

Toothbrush Material

| | Nylon 6,12 | Nylon 6,6 with | |
|---|---|---|---|
| trial | (CFU) | 3% additive (CFU) | % reduction |
| 1 | 290 | 387 | 25 |
| 2 | 980 | 1192 | 18 |

All the references discussed in this application are incorporated by reference in their entirety for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

1. A material useful in oral care products which comprises a silicon-modified polyamide blended with a different polyamide material.

2. The material as claimed in claim 1, wherein said different polyamide material is a synthetic polyamide.

3. The material as claimed in claim 1, wherein said different polyamide material is nylon 6, nylon 6,6; nylon 6,10 or nylon 6,12.

4. The material as claimed in claim 1, wherein said different polyamide material is nylon 6,6.

5. The material as claimed in claim 1, wherein said siloxane-based polyamide (1) comprise both siloxane groups and amide groups to thicken compositions containing silicone fluids (volatile and/or non-volatile silicone fluids); (2) are non-flowable solids at room temperature; and (3) dissolve in a fluid which contains silicone at a temperature of 25-160 degrees C. to form a translucent or clear solution at a temperature in this range.

6. The material as claimed in claim 1, wherein the silicon-modified polyamide comprises
a) 0.5-80 percent by weight based on the total weight of the composition of at least one siloxane-based polyamide;
(b) 5-95 percent by weight silicone fluid; and
(c) if (a)+(b) do not equal 100 percent, then a portion of solvent sufficient to make 100 percent, wherein:
the siloxane-based polyamide is formed from units of Formula A 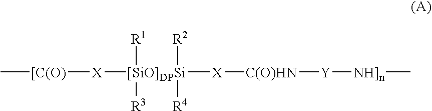
where: (1) n is a number selected from the group consisting of 1-500, where n is the number of units in the polyamide;
(2) DP is an average value for degree of polymerization of a siloxane portion of the polyamide and is selected from the group consisting of 1-700;
(3) X is a linear or branched chain alkylene having 1-30 carbons;
(4) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl; and
(5) Y is selected from the group consisting of linear or branched chain alkylenes having 1-40 carbons, wherein the alkylene group itself may optionally be substituted by at least one member selected from the group consisting of
(i) hydroxy;
(ii) C₃-C₈ cycloalkane;
(iii) 1-3 members selected independently from the group consisting of C₁-C₃ alkyls; phenyl optionally substituted by 1-3 members independently selected from the group consisting of C₁-C₃ alkyls;
(iv) C₁-C₃ alkyl hydroxy; and
(v) C₁-C₆ alkyl amine;
or Y may be Z where Z=R²⁰ T(R²²)R²¹ where each of R²⁰, R²¹ and R²² is independently selected from the group consisting of linear and branched C₁-C₁₀ alkyl or alkylene groups; and T is selected from the group consisting of CR, where R is selected from the group consisting of hydrogen, the group consisting of the group defined for R¹-R⁴; and a trivalent atom selected from the group consisting of N, P and Al; and wherein each value for X, Y, DP and R¹-R⁴ may be the same or different for each unit in the polyamide.

7. The material as claimed in claim 6, wherein said different polyamide material is nylon 6, nylon 6,6; nylon 6,10 or nylon 6,12.

8. The material as claimed in claim 6, wherein said different polyamide material is nylon 6,6.

9. A filament which comprises said material as claimed in claim 1.

10. A filament which comprises said material as claimed in claim 8.

11. An oral care product which comprises at least one of said filament as claimed in claim 9.

12. The oral care product as claimed in claim 11, wherein the product is a toothbrush, dental floss, dental tape or paint brush.

13. Dental floss which comprises at least one of said filaments as claimed in claim 9.

14. A toothbrush bristle which comprises at least one of said filaments as claimed in claim 10.

15. A toothbrush which comprises at least one toothbrush bristle as claimed in claim 13.

16. The toothbrush as claimed in claim 15, wherein said toothbrush is an electric toothbrush.

17. The toothbrush as claimed in claim 15, wherein said toothbrush is a non-electric toothbrush.

18. A brush used for whitening the teeth which comprises at least one of said filaments as claimed in claim 9.

19. Dental tape which comprises at least one of said filaments as claimed in claim 9.
